(19) United States
(12) Patent Application Publication
(10) Pub. No.: US 2010/0057088 A1
(43) Pub. Date: Mar. 4, 2010

(54) Distal Femoral Cutting Guide

(75) Inventor: Asit Shah, Ridgewood, NJ (US)

Correspondence Address: Ryder, Lu, Mazzeo & Konieczny LLC, PO Box 387, CONSHOHOCKEN, PA, 19428-0387, US

(73) Assignee: Maxx Orthopedics, Inc., Plymouth Meeting, PA (US)

(21) Appl. No.: 12/198,439

(22) Filed: Aug. 26, 2008

(51) Int. Cl. A61B 17/58; A61F 5/00

(52) U.S. Cl. 606/88; 606/87

(57) ABSTRACT

An apparatus for resecting the distal face of either the left or right femur at a predetermined valgus angle relative to the patient's intramedullary canal prior to implanting the femoral component of a total knee prosthesis. The apparatus has a distal elongate sword having a longitudinal axis and being adapted for insertion into the intramedullary canal of the femur. A proximal handle is connected to and has a longitudinal axis coaxial with the sword. A base cartridge is fixed intermediate the sword and handle and has an axial passage extending therethrough. A face plate is fixed at the distal end of the base cartridge and is adapted to abut the face of the natural distal femur. The face plate is oriented at the predetermined valgus angle relative to the longitudinal axis of the sword. A cutting jig connects to the base cartridge. The cutting jig has a guide plate with at least one blade slot adapted to receive and guide a cutting blade, and a bracket that supports the guide plate and detachably engages the axial passage and orients the at least one blade slot at the predetermined valgus angle relative to the longitudinal axis.

### FIELD OF THE INVENTION

The invention relates to an apparatus and method for resecting the distal face of the femur at a predetermined valgus angle relative to the patient's intramedullary canal prior to implanting the femoral component of a total knee prosthesis.

### BACKGROUND OF THE INVENTION

Total knee replacement surgery is well-known in the prior art and is becoming more commonplace every day. Total knee replacement surgery involves implantation of complimenting articulating femoral and tibial components to the distal femur and proximal tibia, respectively. In order to properly affix such components, the femur and tibia must be resected to conform to the size and shape of the respective prosthetic component.

To implant the femoral component, the distal face of the femur must be resected to form a flat, transverse facet or face that is parallel to the ground when the patient stands upright so that the joint is loaded evenly. It is known that the femur does not extend upwardly at a perfectly normal angle relative to the ground; rather, it projects outwardly at a valgus angle relative to the anatomic axis. Therefore, the face of the distal femur must be cut at angle relative to the longitudinal axis of the femur so that the resected face is co-planar (parallel) to the ground. For example, it is known in the prior art to resect the face of the femur at a 6 degree valgus angle.

A number of patents have issued that disclose instruments and/or jigs for resecting the distal femur. Many of the devices include a cutting block that references an intramedullary rod inserted in the intramedullary canal of the femur. To accommodate the asymmetrical shape of the distal femur, such devices have complex designs that require time-consuming reconfiguration when the device is switched from the left to the right femur, and vice versa. Alternatively, the surgeon may use separate devices that are specially configured for use on either the left femur or the right femur. However, the added cost of purchasing, maintaining and sterilizing two resection devices is undesirable. Therefore, it would be desirable to provide a single device that can be used to resect both the left and right femur, which does not require complex reconfiguration.

### SUMMARY OF THE INVENTION

The invention comprises an apparatus for resecting the distal face of the femur at a predetermined valgus angle relative to the patient's intramedullary canal prior to implanting the femoral component of a total knee prosthesis. The apparatus can be used for resecting either the left or right femur. The apparatus has a first operable orientation adapted for resecting the left femur and a second operable orientation offset 180 degrees from the first operable position for resecting the right femur.

The apparatus includes a distal elongate sword having a longitudinal axis and being adapted for insertion into the intramedullary canal of the femur. A proximal handle is connected to the sword and has a longitudinal axis coaxial with the sword. A base cartridge is fixed intermediate the sword and handle and has an axial passage extending therethrough. The axial passage has first and second surface openings that are radially spaced 180 degrees from one another. The first surface opening is oriented vertically upwardly in the first operable position and the second surface opening is oriented vertically upwardly in the second operable position.

A face plate is fixed at the distal end of the base cartridge and is adapted to abut the face of the natural distal femur. The face plate is oriented at the predetermined valgus angle relative to the longitudinal axis of the sword.

A cutting jig connects to the base cartridge. The cutting jig has a guide plate with at least one blade slot adapted to receive and guide a cutting blade. A bracket supports the guide plate and detachably engages the axial passage in the base cartridge. The bracket also orients the blade slots at the predetermined valgus angle relative to the longitudinal axis. Each of the cutting jig and the guide plate has a symmetrical construction about a central sagital plane. The cutting jig is properly aligned at the predetermined valgus angle to resect the left femur when the jig engages the axial passage through the first surface opening, and is properly aligned at the predetermined valgus angle to resect the right femur when the jig engages the axial passage through the second surface opening.

The guide plate can rotate on said support bracket. The axis of rotation of said guide plate is parallel to the longitudinal axis of said apparatus.

The guide plate includes means for temporarily affixing the guide plate to the patient's femur. The bracket can be disconnected from the guide plate and the sword removed from the intramedullary canal after the guide plate it is temporarily affixed to the patient's femur.

### BRIEF DESCRIPTION OF THE DRAWINGS

FIG. 1 is a perspective of an apparatus in accordance with an embodiment of the invention;

FIG. 2 is an exploded assembly drawing of the apparatus of FIG. 1.

FIG. 3 is a side elevation of the apparatus of FIG. 1.

FIG. 4 is top plan view of the apparatus of FIG. 1;

FIG. 5 is an axial cross-section of the handle of the apparatus shown in FIG. 1;

FIG. 6 is a top plan view of the cartridge of FIG. 1, shown at a first angular position;

FIG. 7 is a top plan view the cartridge of FIGS. 1 and 6, shown at a second angular position axially rotated 90 degrees;

FIG. 8 is a top plan view of the cartridge of FIGS. 1, shown at a third angular position axially rotated 180 degrees;

FIG. 9 is a perspective of the cutting jig of the apparatus shown in FIG. 1;

FIG. 10 is a back elevation of the guide plate of the cutting jig shown in FIG. 9.

FIG. 11 is a top plan view of the face plate shown in FIG. 1;

FIG. 12 is a cross section taken along lines 12-12 of FIG. 11;

FIG. 13 is a side elevation of the sword portion of the apparatus shown in FIG. 1;

FIG. 14 is a perspective of a retraction handle that removably connects with the proximal end of the handle of the apparatus shown in FIG. 1.

### DETAILED DESCRIPTION OF PREFERRED EMBODIMENTS

For the purpose of illustrating the invention, an embodiment of the invention is shown in the accompanying drawings. However, it should be understood by those of ordinary skill in the art that the invention is not limited to the precise arrangements and instrumentalities shown therein and described below. Throughout the specification, like reference numerals are used to designate like elements. Numerous changes and modifications within the spirit and scope of the invention will become apparent to those skilled in the art from this detailed description.

Unless otherwise defined, all technical and scientific terms used herein in their various grammatical forms have the same meaning as commonly understood by one of ordinary skill in the art to which this invention belongs. The terms anterior, posterior, proximal, distal, medial, lateral, sagital, coronal, and transverse are used herein with their conventional medical/anatomical meaning as defined, for example, in Dorland's Illustrated Medical Dictionary. When used in connection with the apparatus, the terms proximal and distal reference the surgeon using the apparatus. In contrast, when used in connection with the femur with which the apparatus is used, the terms proximal, distal, anterior and posterior reference the patient. The term valgus angle means the angle formed between the anatomic axis and the longitudinal axis of the femur.

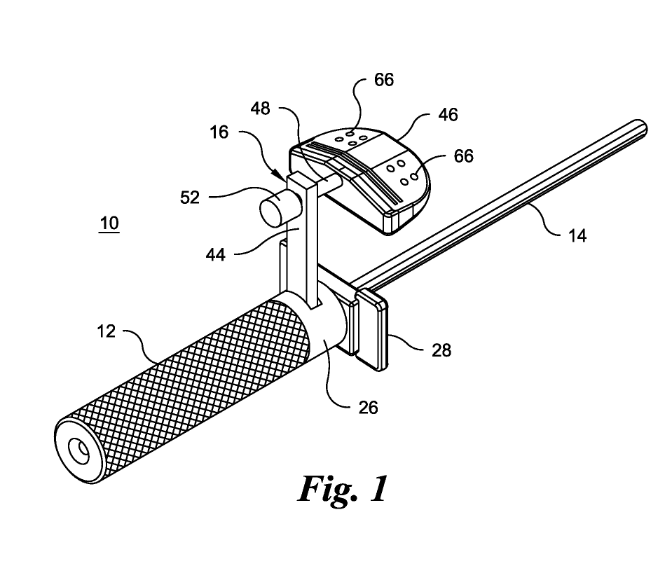

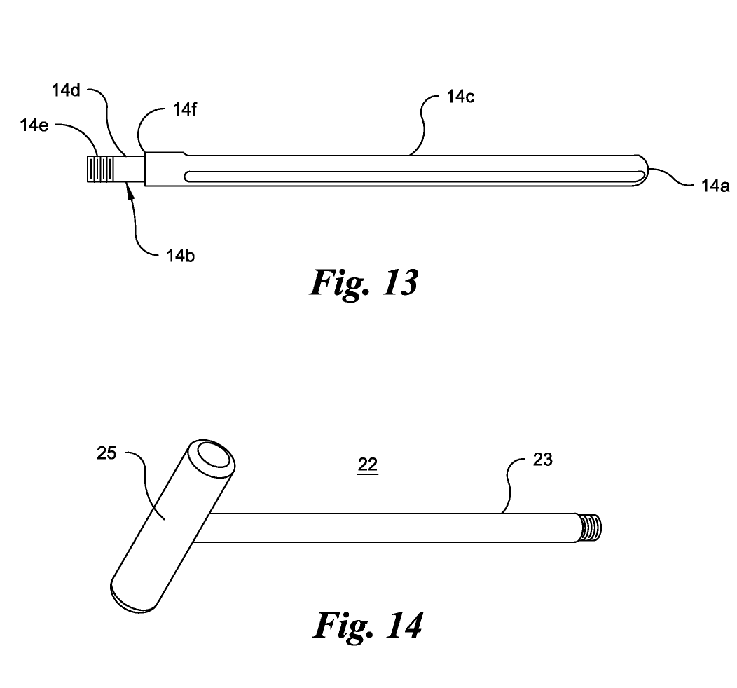

A distal femoral cutting apparatus in accordance with an embodiment of the invention is illustrated in FIGS. 1-14 and is designated generally by reference numeral 10. The apparatus 10 guides the saw blade during resection of the distal face of the femur at a predetermined angle relative to the patient's intramedullary canal prior to implanting the femoral component of a total knee prosthesis. The predetermined angle is preferably equal to the valgus angle of the patient's femur so that the transverse resected face of the femoral component is parallel to the ground surface.

The apparatus has an elongate handle 12 and sword 14 that are coaxial. A base cartridge 26, face plate 28, and cutting jig 16 are fixed intermediate the handle 12 and the sword 14. In a preferred embodiment, the handle 12, sword 14, base cartridge 26 and face plate 28 are connected by mating threaded connectors so that the various components can be interchanged with similar components having different physical properties such as length and angular orientation. The components could have different connectors or, alternatively, could be integrally formed or permanently fixed to one another.

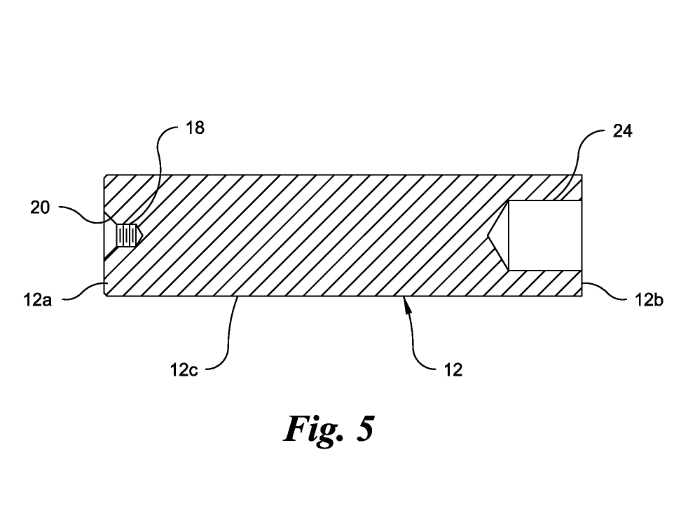

The handle 12 generally comprises an elongate, cylindrical bar. The diameter and length of the handle 12 are selected for comfort and control by the surgeon. For example, in one embodiment, the diameter is about 1¼ in. and the length is about 5 in. Referring to FIG. 5, the handle 12 has a proximal end 12a, a distal end 12b, and an annular gripping surface 12c. In a preferred embodiment, the annular surface 12c is knurled for better grip. The proximal end 12a has a threaded bore 18 with a chamfered side wall 20. The first threaded bore 18 is adapted to connect with the stem 23 of an extractor handle 22, described below and shown in FIG. 14. The distal end 12b also has a threaded bore 24, which connects to the base cartridge 26. The first 18 and second 24 bores are preferably coaxial with the central, lengthwise axis of the handle 12.

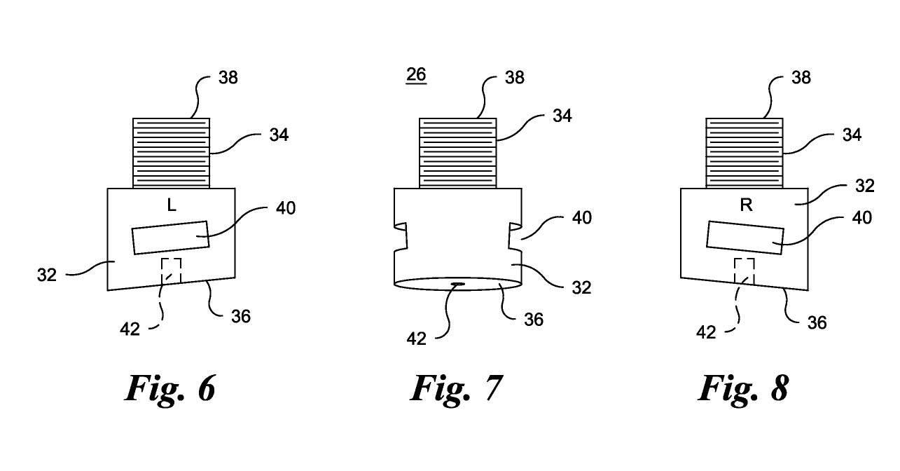

The cartridge 26 provides means for mounting the cutting jig in a first orientation for resecting the left femur, and in a second orientation for resecting the right femur. In a preferred embodiment, the cartridge 26 comprises a cylindrical post having a first section 32 and a second reduced-diameter section 34, which are coaxial on a central longitudinal axis as seen in FIGS. 6-8. The radii of the first and second sections are constant about the central axis. Both axial ends 36, 38 of the cartridge are flat. However, the end 36 of the first section 32 is skew to the central axis while the end 38 of the second section 34 is perpendicular to the central axis. The plane of the first end 36 forms an angle with the central axis preferably equal to the predetermined valgus angle.

The annular surface of the second section 34 of the cartridge 26 is threaded to engage the threaded bore 24 in the distal end 12b of the handle 12. The annular surface of the first section 32 of the cartridge is preferably smooth and has a diameter about equal to the diameter of the handle 12. The first section 32 has a channel 40 extending radially therethrough. In the embodiment shown in FIGS. 6-8, the channel 40 has a rectangular cross-section; however, it should be appreciated that the channel 40 could have a different cross section without departing from the scope of the invention. As best seen in FIG. 6, the central axes of the channel 40 are offset from the longitudinal axis of the cartridge 26 by the same angle as the first tapered radial end 36 of the cartridge 26.

A threaded axial bore 42 is formed in the first tapered end 36 of the cartridge. The bore 42 is coaxial with the central axis of the cartridge 26. The threaded bore 42 engages the proximal end 14b of the sword 14 and aligns the sword 14 coaxial with the longitudinal axis of the handle 12.

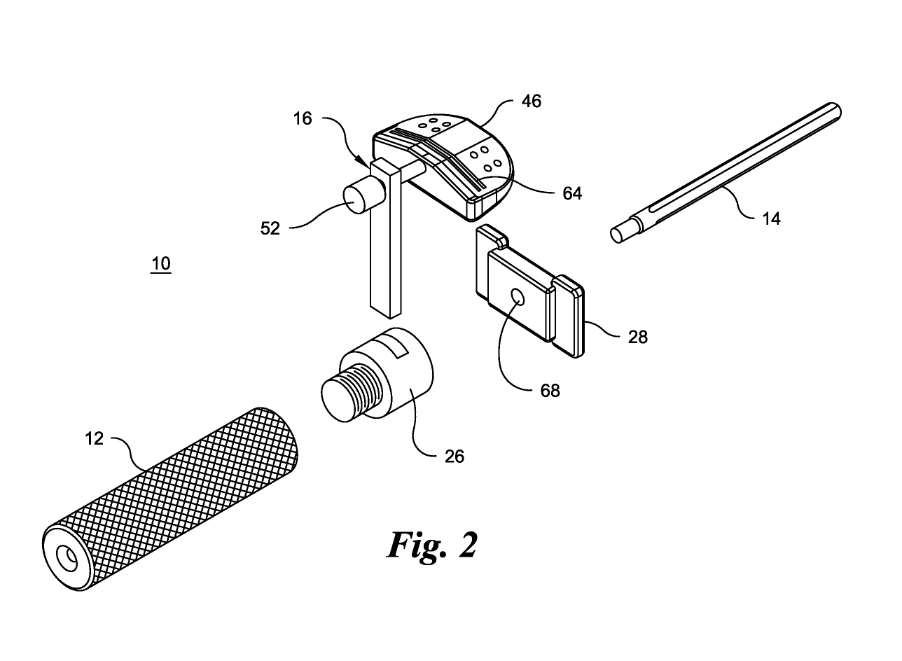

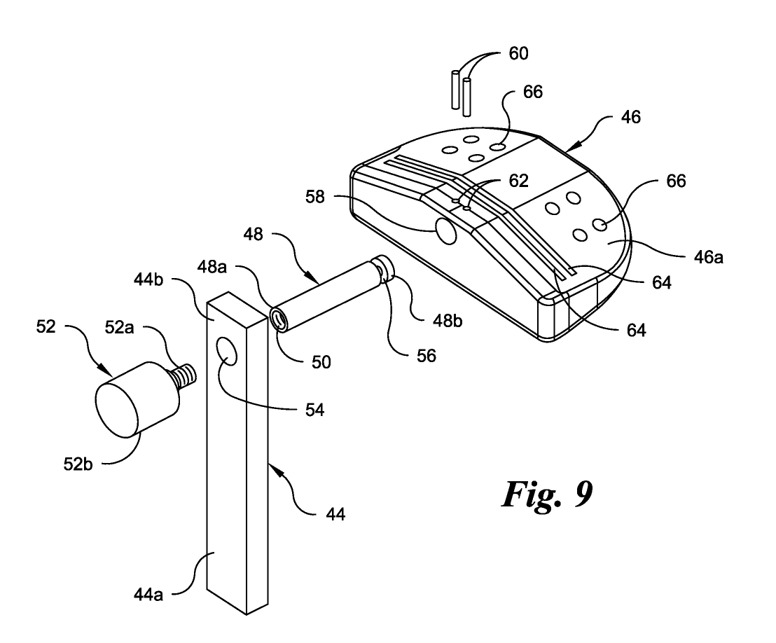

Referring to FIGS. 2 and 9, the cutting jig 30 includes a guide plate 46 vertically supported above the handle 12 by a support bracket. In a preferred embodiment, the support bracket comprises a vertical support post 44 connected to a horizontal support rod 48. The lower end 44a of the support post 44 nests in the channel 40 of the cartridge 26. The support post 44 has the same rectangular cross section and slightly smaller dimensions as the channel 40 so that the post 44 fits snugly but can slide within the channel 40. The support post 44 extends vertically upwardly relative to the longitudinal axis of the apparatus 10 when the sword 14 is inserted into the intramedullary canal of the patient. The plane of the distal face 44c of the support post 44 is not normal to the longitudinal axis of the cartridge 26 but rather offset at the predetermined valgus angle. As described below, the support post 44 can be raised or lowered within the channel 40 to engage and disengage the guide plate 46 relative to the anterior surface of the femur.

The proximal end 48a of the support rod 48 is removably connected to the upper end 44b of the support post 44. The proximal end 48a of the support rod 48 has a threaded axial bore 50, which engages a thumb screw 52. To secure the support rod 48 to the post 44, the threaded shank 52a of the thumb screw extends through a bore 54 in the upper end 44b of the support post 44 and engages the bore 50 in the post 44. The head 52b of the thumb screw has a larger diameter than the bore 54 and preferably has a knurled surface.

The distal end 48b of the support rod 48 connects to the guide plate 46 through a bore 58 in the proximal face 46c of the guide plate 46. To create a rotatable connection therebetween, two dowels 60 engage and straddle an annular groove 56 in the distal end 48b of the support rod. The dowels 60 extend through two dowel bores 62, which are oriented downwardly from and orthogonal to the upper face 46a of the guide plate 46. In a preferred embodiment, the dowels 60 are welded in place.

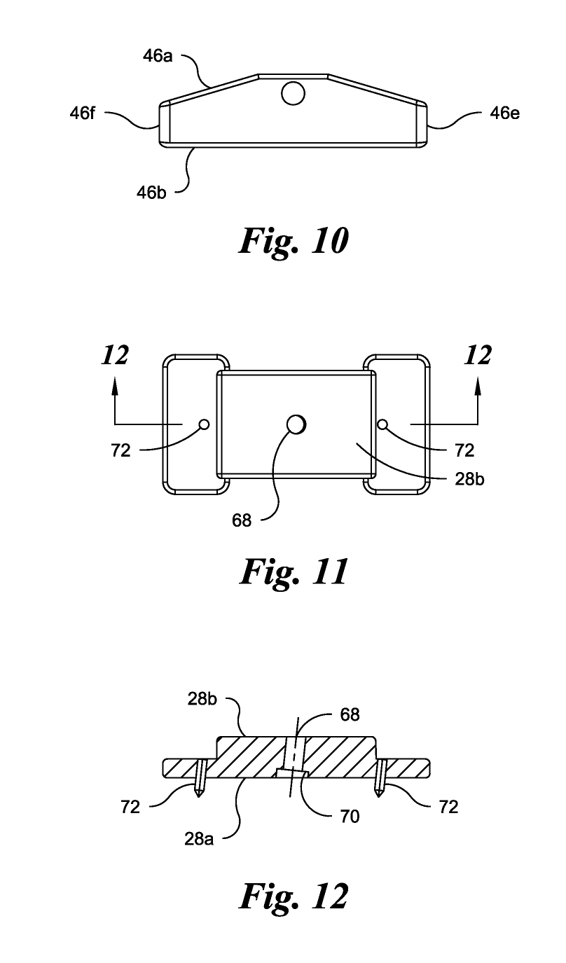

The guide plate 46 has a generally planar lower face surface 46b. The upper face surface 46a has an irregular shape as shown by the back elevation of FIG. 10. A pair of cutting guide slots 64 extends through the guide plate 46. The slots 64 are oriented widthwise (medial/lateral) from one side 46e to the other side 46f and parallel to distal face 44c of the support post 44. As a result, the slots 64 are also arranged at the predetermined valgus angle relative to the longitudinal axis of the apparatus 10.

The parallel slots 64 are spaced along the longitudinal axis of the apparatus 10 to provide two different amounts of resection. For example, in the embodiment shown in FIGS. 9 and 10, the proximal slots are axially, distally located 8 mm and 11 mm from the distal face of the face plate 28 so that either 8 mm or 11 mm of femur is resected. A plurality of pin holes 66 are symmetrically spaced on the guide plate 46. During resection, the guide plate 46 is pinned to the femur by driving pinning nails through the pin holes 66 and into the femur.

The sword 14 and face plate 28 are connected to the tapered, distal end 36 of the cartridge 36. The face plate 28 has a planar distal face 28a and a stepped proximal face 28b. Referring to FIGS. 11 and 12, the central portion of the proximal face 28b is elevated relative to the outer portions and abuts the tapered radial end 36 of the cartridge. The planes of the distal face 28a and proximal face 28b are parallel to one another and parallel to the distal face 44c of the post 44 when connected to the tapered distal end 36 of the cartridge, and are therefore also arranged at the predetermined valgus angle.

A central bore 68 extends through the center of the face plate 28. An enlarged countersink 70 in the distal surface 28a is coaxial with the bore 68. The central axis of the bore 68 is skew to the distal 28a and proximal 28b faces. Preferably, the bore is offset by an angle equal to the predetermined valgus angle so that the sword 14 will align with the central axis of the handle 12 when attached. A pair of stabilizing pins 72 flank the central bore and extend from the distal face 28a. In a preferred embodiment, the pins 72 are located on the transverse central axis of the face plate 28 as shown in FIG. 11. The pins 72 also extend skew to the distal face 28a of the face plate 28. Preferably, the pins 72 are offset by an angle equal to the predetermined valgus angle.

The sword 14 comprises an elongate rod having a rounded tip 14a at the distal end, a reduced-diameter shank 14b at the proximal end, and a fluted intermediate annular surface 14c. The flutes allow marrow to channel out of the intramedullary canal when the sword 14 is inserted. The shank 14b has a threaded portion 14e and a non-threaded portion 14d. The non-threaded portion 14d engages the central bore 68 in the face plate 28. The threaded portion 14e engages the threaded bore 42 in the distal end of the cartridge 26. The shoulder 14f of the sword 14 abuts the shoulder of the countersink 70 in face plate 28 and secures the face plate 28 to the cartridge when the sword 14 is tightened. Since the apparatus is modular and can be easily disassembled, swords having different physical sizes and shapes can be interchanged. For example, a shorter sword can be used to accommodate Asian patients, who have a generally shorter femoral canal compared to Caucasians.

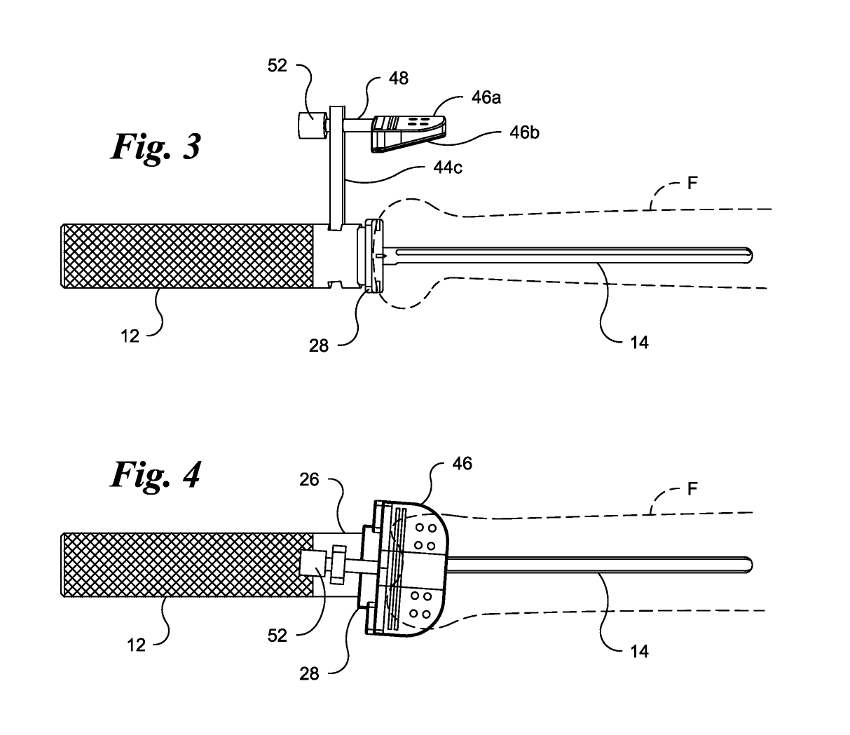

The apparatus 10 of the present invention is shown in FIGS. 3 and 4 in a first operable orientation on the distal femur F of a patient after the patient's knee cap has been averted and the marrow has been evacuated from the intramedullary canal through a hole in the distal face. Initially, the sword 14 of the apparatus 10 is inserted into the intramedullary canal as shown in FIGS. 3 and 4. During insertion, the surgeon radially aligns the apparatus 10 so that the appropriate channel opening (left or right) is aligned with the top dead center of the canal. The sword 14 is inserted until the face plate 28 contacts the distal femoral condyles. The surgeon then taps or urges the handle 12 forward so that the stabilizing pins 72 embed into the condyles to prevent axial rotation of the sword 14.

Next, the surgeon inserts the lower end 44a of the support post 44 into the channel 40 in the cartridge 26. The post 44 slides downward until the lower face 46b of the guide plate 46 contacts the anterior surface of the condyles. If the anterior surface is irregular, the surgeon rotates the guide plate 46 about the support rod 48 until the lower face 46b rests flatly on both condyles. The guide plate 46 is then pinned to the condyles by driving pinning nails through pin holes 66. The sword is then removed from the patient's knee by disconnecting the support rod 48 from the support post 44 and withdrawing the sword 14 from the intramedullary canal. With the guide plate 46 fixed in place and the sword 14 removed, the surgeon resects the femur using the slots 64 to guide the blade of the saw. When the resection is complete, the pinning nails are extracted and the guide plate removed from the femur.

The above-described procedure can be repeated on the opposite knee with no reconfiguration of the apparatus. To resect the opposite knee, the apparatus 10 must simply be rotated to the second operable orientation so that appropriate channel opening (left or right) is aligned with the top dead center of the canal. With reference to the first operable orientation, the second operable orientation is radially offset 180 degrees. In the second operable orientation, the face plate 28 and the slots 64 of the guide plate are oriented at the opposite valgus angle as needed for the opposite knee. In other words, if the valgus angle of the resected face on the first knee was +6 degrees, the valgus angle of the resected face on the second knee will be −6 degrees simply by axially rotating the handle 180 degrees prior to insertion into the intramedullary canal. Thus, a single apparatus 10 can be used to resect either the left or right femur without reconfiguring or mechanically adjusting the apparatus, and without providing specific left or right adapters.

In the embodiment described above, the apparatus is designed with a valgus angle of 6 degrees. However, the apparatus could be designed with a different valgus angle by changing the offset angle of the axial bore in the cartridge.

Although it is believed that a surgeon generally resects the femur using the same valgus angle for most if not all patients, the apparatus can be provided with multiple cartridges having axial bores offset at different valgus angles. In this embodiment, the surgeon can change the valgus angle from patient to patient simple by disassembling the device prior to surgery and substituting a cartridge having the desired valgus angle.

During the normal procedure, it may be difficult to remove the sword 14 prior to resecting the femur. If this occurs, the surgeon can install the extractor handle 22 on the handle 12 by simply screwing the threaded end of the shank 23 into the threaded bore 18 in the proximal end of the handle 12. The surgeon can then additionally pull on the T-shaped grip 25 along with the handle 12. Alternatively, the surgeon can tap on the T-shaped grip 25 with a hammer to jar the sword 14 loose.

1. An apparatus for resecting the distal face of the femur at a predetermined valgus angle relative to the patient's intramedullary canal prior to implanting the femoral component of a total knee prosthesis, comprising:
a) a distal elongate sword having a longitudinal axis and being adapted for insertion into the intramedullary canal of the femur;
b) a proximal handle connected to and having a longitudinal axis coaxial with said sword;
c) a base cartridge being fixed intermediate said sword and handle and having an axial passage extending therethrough;
d) a face plate at the distal end of the base cartridge adapted to abut the face of the natural distal femur, said plate oriented at the predetermined valgus angle relative to the longitudinal axis of the sword; and,
e) a cutting jig having: (i) a guide plate with at least one blade slot adapted to receive and guide a cutting blade; and, (ii) a bracket that supports said guide plate and detachably engages said axial passage and orients said at least one blade slot at the predetermined valgus angle relative to the longitudinal axis;
wherein said apparatus can be attached to and used for resecting either the left or right femur.

2. The apparatus recited in claim 1, wherein said apparatus has a first operable orientation adapted for resecting the left femur and a second operable orientation offset 180 degrees from the first operable position for resecting the right femur.

3. The apparatus recited in claim 1, wherein said cutting jig has a symmetrical construction about a central sagital plane.

4. The apparatus recited in claim 1, wherein said guide plate has a symmetrical construction about a central sagital plane.

5. The apparatus recited in claim 2, wherein said axial passage has first and second surface openings that are radially spaced 180 degrees from one another.

6. The apparatus recited in claim 5, wherein the cutting jig is properly aligned at the predetermined valgus angle to resect the left femur when the jig engages the axial passage through the first surface opening, and is properly aligned at the predetermined valgus angle to resect the right femur when the jig engages the axial passage through the second surface opening.

7. The apparatus recited in claim 6, wherein the first surface opening is oriented vertically upwardly in the first operable position and the second surface opening is oriented vertically upwardly in the second operable position.

8. The apparatus recited in claim 1, wherein said guide plate includes means for temporarily affixing the guide plate to the patient's tibia.

9. The apparatus recited in claim 8, wherein said bracket can be disconnected from said guide plate and the sword removed from the intramedullary canal after said guide plate it is temporarily affixed to the patient's tibia.

10. The apparatus recited in claim 1, wherein said guide plate can rotate on said support bracket.

11. The apparatus recited in claim 10, wherein the axis of rotation of said guide plate is parallel to the longitudinal axis of said apparatus.

12. An apparatus for resecting the distal face of the femur at a predetermined valgus angle relative to the patient's intramedullary canal prior to implanting the femoral component of a total knee prosthesis, comprising:
a) an elongate rod having a distal sword portion for insertion into the intramedullary canal of the femur and a proximal handle portion, said rod having a longitudinal axis that aligns with the patient's intramedullary canal;
b) a cutting jig having a support bracket and a guide plate with at least one blade slot adapted to receive and guide a cutting blade;
c) means for mounting the cutting jig on said rod in a first orientation wherein the guide plate is arranged to resect the left femur at the predetermined valgus angle, and for mounting the cutting jig on said rod in second orientation wherein the guide plate is arranged to resect the right femur at the predetermined valgus angle.

13. The apparatus recited in claim 12, including a face plate at the distal end of said mounting means adapted to abut the face of the natural distal femur, said plate oriented at the predetermined valgus angle relative to the longitudinal axis of the sword.

14. The apparatus recited in claim 12, wherein said cutting jig has a symmetrical construction about a central sagital plane.

15. The apparatus recited in claim 12, wherein said guide plate includes means for detachably fixing said guide plate the femur, and said support bracket is removably connected to said guide plate.

16. The apparatus recited in claim 12, wherein said mounting means comprises an aperture in said handle, and said bracket comprises a post.

17. The apparatus recited in claim 12, including a retraction handle that is releasably connectable to the handle portion of said rod.

18. The apparatus recited in claim 12, wherein said guide plate can rotate on said support bracket.
